(19) United States
(12) Patent Application Publication
(10) Pub. No.: US 2011/0192396 A1
(43) Pub. Date: Aug. 11, 2011

(54) PHARMACEUTICAL COMPOSITION FOR PREVENTING OR TREATING CHRONIC OBSTRUCTIVE PULMONARY DISEASE

(75) Inventors: Sun Mee Yang, Yongin-si (KR); Kwon Jo Lim, Seoul (KR); Hyun Jin Park, Suwon-si (KR); Il Hwan Kim, Cheongwon-gun (KR); Sang Ho Lee, Yongin-si (KR); Jong Wook Lee, Yongin-si (KR)

(73) Assignee: DAEWOONG CO., LTD., Seongnam-si (KR)

(21) Appl. No.: 13/123,481

(22) PCT Filed: Oct. 9, 2009
(86) PCT No.: PCT/KR2009/005770

§ 371 Date: Apr. 8, 2011

(30) Foreign Application Priority Data
Oct. 9, 2008 (KR) 10-2008-0099336

(51) Int. Cl. A61M 11/00; A61K 38/22; A61P 11/00; A61P 11/08; A61M 15/00

(52) U.S. Cl. 128/200.14; 514/9.6; 128/203.12

(57) ABSTRACT

Provided is a pharmaceutical composition for preventing or treating mucus hypersecretion in airways or chronic obstructive pulmonary diseases (COPDs), the pharmaceutical composition including an epidermal growth factor (EGF) and a pharmaceutically acceptable carrier. In addition, a combined preparation capable of respectively administering the pharmaceutical composition and chemotherapeutics is provided.

### TECHNICAL FIELD

The present invention relates to a pharmaceutical composition for preventing or treating chronic obstructive pulmonary diseases, and more particularly, to a pharmaceutical composition for preventing or treating mucus hypersecretion in airways or chronic obstructive pulmonary diseases, the pharmaceutical composition including an epidermal growth factor (EGF) as an active ingredient.

### BACKGROUND ART

Chronic obstructive pulmonary disease (COPD) is caused by direct/indirect smoking, occurs in occupational groups that handle environmental pollutants such as cadmium (Cd) and silica (SiO₂), and in rare instances is caused by abnormal gene α1-antitrypsin.

When lungs are inflamed, mucus is overproduced in order to protect lung tissue from the inflammation. COPD is a fatal disease caused by impairment of mucus clearance, thereby obstructing airways and destructing alveolar cells which leads to hypoxia, thereby causing death.

COPD is classified into 2 groups according to the location of lesions and mechanism: chronic bronchitis and emphysema. Chronic bronchitis is clinically characterized by airway obstruction due to accumulated mucus that is overproduced in airway epithelium cells but not removed. Emphysema is clinically characterized by hypoxia, cardiac diseases, or irreversible bronchiole obstruction due to destructed alveolar cells. In particular, in emphysema, alveolar cells are irreversibly destructed due to hydrolysis by elastase activated by inflammatory cells, so that the number of CD⁸⁺, T lymphocyte, macrophage, and neutrophil is increased from a tissue pathological point of view.

In general, mucus of airways in a human body facilitates the removal of particles or inflammatory factors inhaled through the airways. However, hypersecretion of mucus may cause a progressive airway obstruction. Coughing cannot remove mucus in peripheral airways. Furthermore, narrow airways including a lot of goblet cells may be easily obstructed particularly by mucus. Thus, there is a need to prevent and treat mucus hypersecretion occurring not only in chronic bronchitis, as one of the COPDs, but also in a variety of pulmonary diseases, such as brochial dilatation, cystic fibrosis, and acute asthma.

EGF is a protein promoting the growth of epidermal cells, and effects thereof on treating wounds such as regeneration of epidermal cells and promotion of cell division have been widely known (U.S. Pat. No. 6,589,540, et. al.). However, there has been no report disclosing effects of EGF on preventing or treating COPDs or mucus hypersecretion in airways.

### DISCLOSURE OF INVENTION

### Technical Problem

While searching for pharmaceuticals used to prevent or treat chronic obstructive pulmonary diseases (COPDs) or mucus overexpression in airways, the present inventors found that an epidermal growth factor (EGF) may be used not only to prevent and treat COPDs including chronic bronchitis and emphysema but also to suppress mucus overexpression in airways to prevent and treat mucus hypersecretion in airways, which is a chronic problem in a variety of pulmonary diseases.

The object of the invention is to provide a pharmaceutical composition for preventing or treating mucus hypersecretion in airways.

The object of the invention is also to provide a pharmaceutical composition for preventing or treating chronic obstructive pulmonary diseases (COPDs).

The object of the invention is also to provide a package for preventing or treating mucus hypersecretion in airways, the package including an inhaler device and a fluidable preparation that is contained in the inhaler device.

### Solution to Problem

According to an aspect of the present invention, there is provided a pharmaceutical composition for preventing or treating mucus hypersecretion in airways, the pharmaceutical composition including an epidermal growth factor (EGF) and a pharmaceutically acceptable carrier.

According to another aspect of the present invention, there is provided a pharmaceutical composition for preventing or treating a chronic obstructive pulmonary disease (COPD), the pharmaceutical composition including an epidermal growth factor (EGF) and a pharmaceutically acceptable carrier.

According to another aspect of the present invention, there is provided a package for preventing or treating mucus hypersecretion in airways, the package including an inhaler device and a fluidable preparation including an epidermal growth factor (EGF) that is contained in the inhaler device and is pharmaceutically active.

### Advantageous Effects of Invention

As described above, the present invention provides a pharmaceutical composition for preventing or treating COPDs or mucus hypersecretion in airways, the pharmaceutical composition including an EGF as an active ingredient. In addition, a preparation including EGF may be contained in an inhaler device to be efficiently used to prevent or treat mucus hypersecretion in airways.

### BRIEF DESCRIPTION OF DRAWINGS

The above and other features and advantages of the present invention will become more apparent by describing in detail exemplary embodiments thereof with reference to the attached drawings in which:

FIG. 1 is a graph illustrating relative amounts of MUC5AC gene expression in NCI-H292 cells treated with PA-LPS and TGF-α for 6 hours to overexpress the MUC5AC gene, and then treated with 50 and 100 ng/mL of EGF, measured using a real time PCR, and the amount of MUC5AC gene expression in a control group treated with PA-LPS and TGF-α only;

FIG. 2 illustrates relative amounts of ICAM-1 gene expression in MLE-12 cells treated with 0.1 U/mL of elastase and 100 ng/mL of EGF, measured using a real time PCR, and the amount of ICAM-1 gene expression in control groups treated with elastase only and media only;

FIG. 3 illustrates the degree of cell toxicity reduction in MLE-12 cells treated with 2 U/mL of elastase and EGF having different concentrations for 24 hours and dyed with neutral red in comparison with those of control groups treated with elastase only and media only;

FIGS. 4, 5, and 6 illustrate optical microscopic images (×40) of tracheas isolated from a group without emphysema (FIG. 4), a control group with emphysema and excipient (FIG. 5), and a group with emphysema treated with 0.1 mg/kg of EGF (FIG. 6) in a PPE-induced rat emphysema model to identify effects of EGF on relieving lesions (B: bronciole, (*): enlargement and breakdown of alveoli);

FIG. 7 shows tissue pathological test results of tracheas and lung in a PPE-induced rat emphysema model to identify effects of EGF on reliving lesions; and

FIG. 8 shows details of the results of FIG. 7.

### MODE FOR THE INVENTION

Hereinafter, the present invention will now be described.

The epidermal growth factor (EGF) used herein is 53 amino acid that is known to be synthesized in duodenum or salivary glands of a normal person and expressed in human breast milk. The amino acid sequence of human EGF is as follows:

(SEQ ID NO: 1)
Asn Ser Asp Ser Glu Cys Pro Leu Ser His Asp
Gly Tyr Cys Leu His Asp Gly Val Cys Met Tyr
Ile Glu Ala Leu Asp Lys Tyr Ala Cys Asn Cys
Val Val Gly Tyr Ile Gly Glu Arg Cys Gln Tyr
Arg Asp Leu Lys Trp Trp Glu Leu Arg.

Proteins used in the experiments of the examples of the present invention have the sequence described above. The EGF may also include a non-human EGF sequence that functions as the EGF in human. The EGF may also include a species variant of the EGF, for example, EGF (49-52) of mice, rats, and pigs; bovine EGF disclosed in US Patent Application No. 2003-0059802; a phase-agent chimera (53) of distinct EGF receptor ligand, or the like. The EGF may also be a polypeptide that has the same sequence and activity as a purified natural EGF. In this regard, recombinantly or chemically synthesized peptides and proteins may also be included. The EGF may also include a protein modified by insertion of at least one amino acid into the natural sequence or deletion of at least one amino acid from the natural sequence as long as biological activities of the EGF that are required herein are substantially preserved. If the biological activities of EGF that are required herein are substantially preserved, EGF fragments, peptide analogs, and peptide mimetics may also be used. Muteins of EGF disclosed in U.S. Pat. No. 6,191,106 issued on Feb. 20, 2001 may also be within the range of the EGF used herein.

The present inventors have found that EGF described above has effects on preventing or treating mucus hypersecretion in airways and COPDs.

Thus, one aspect of the present invention provides a pharmaceutical composition for preventing or treating mucus hypersecretion in airways including EGF and a pharmaceutically acceptable carrier.

Another aspect of the present invention also provides a pharmaceutical composition for preventing or treating chronic obstructive pulmonary diseases (COPDs) including EGF and a pharmaceutically acceptable carrier. The COPD may be chronic bronchitis or emphysema.

The EGF may be chemically or recombinantly synthesized EGF, or natural EGF as described above, or preferably EGF isolated from recombinant E. coli (JM101) and purified. In this regard, the EGF is obtained by fermenting recombinant E. coli for 48 to 72 hours using a fed-batch culture, and isolating the supernatant of the fermented broth using Amberchrome CG71 chromatography and Q-sepharose chromatography (U.S. Pat. No. 5,652,120, JP 2609515, EP 0 652 954, KR102993, KR 107023, KR 110123, KR 114856). It has been identified in U.S. Pat. No. 5,652,120, JP 2609515, EP 0 652 954, KR 102993, KR 107023, KR 110123, KR 114856 that the EGF isolated from the recombinant E. coli and purified is the same as the human EGF.

The present inventors identified that the EGF has effects on reducing mucus overexpression, inflammation, and cell death in an in vitro model, and have found that EGF has effects on preventing or treating mucus hypersecretion in airways and COPDs by observing pathological findings in an in vivo model showing COPDs. The in vitro and in vivo experiments are described with reference to the examples described below.

In the pharmaceutical composition, the pharmaceutically acceptable carrier may vary according to the type of formulation based on the administration method of the pharmaceutical composition. The pharmaceutical composition may be administered via intravenous injection, subcutaneous injection, intratracheal injection, or inhalation. Thus, the pharmaceutical composition may be formulated as an injection, a liquid for inhalation, a spray, a suspension, or a colloidal suspension. Saline or a buffer solution may be used as a major pharmaceutically acceptable carrier. If required, the formulation may further include additives such as a viscosity controlling agent, an osmolality adjusting agent, a buffering agent, a pH adjusting agent, a stabilizer, an aromatic, a colorant, and a preservative. Techniques required for the formulation and the pharmaceutically acceptable carrier and additives are obvious to those of ordinary skill in the art (Remington's Pharmaceutical Sciences (19^{th} ed., 1995)).

In the pharmaceutical composition, the EGF may be administered in a therapeutically effective amount for preventing or treating mucus hypersecretion in airways or COPDs, and the therapeutically effective amount may vary according to the severity of mucus hypersecretion in airways or COPDs, and age, gender, and sensitivity of a patient. For example, the pharmaceutical composition may be administered to prevent or treat mucus hypersecretion in airways and COPDs such that 0.1 μg/kg to 100 mg/kg of EGF is administered 1 to 5 times a week for 1 to 8 weeks. The therapeutically effective amount may also vary according to the type of formulations.

The pharmaceutical composition including the EGF may be contained in a device suitable for inhalation to be conveniently used for preventing or treating mucus hypersecretion in airways.

The present invention also provides a package for preventing or treating mucus hypersecretion in airways, the package including an inhaler device and a fluidable preparation including an epidermal growth factor (EGF) contained in the inhaler device.

The inhaler device may be a meter-dose inhaler or a spray, but any device that is commonly used for inhalation may also be used. In the meter-dose inhaler, the EGF may be mixed with a propellant to prepare the fluidable preparation. Any propellant that is commonly used in the art may be used. For example, a hydrocarbon propellant may be used. In the spray, the EGF may be mixed with saline or a buffer solution to prepare the fluidable preparation. When using the spray, the fluidable preparation may be administered to the airway in the form of fine particulate liquid form having substantially uniform fine particle size. The preparation using such an inhaler device is obvious to those of ordinary skill in the art (Remington's Pharmaceutical Sciences (19^{th} ed., 1995)).

Hereinafter, one or more embodiments will be described in detail with reference to the following examples. However, these examples are not intended to limit the purpose and scope of the invention

### Example 1

### Effects of EGF on COPD and Mucus Hypersecretion in Airways

### 1. MATERIALS AND METHODS

1-1. Effects on Suppressing Mucus Expression

NCI-H292 cells (ATCC, CRL-1848) were divided into a 6-well plate such that the number of the NCI-H292 cells per well was 10⁶. The NCI-H292 cells were incubated in a 5% CO₂ incubator at 37° C. for one day. The NCI-H292 cells were incubated in a serum free medium for one day, and then treated with 100 mg/mL of Pseudomonas aeruginosa lipopolysaccharide (PA-LPS, Sigma L8643) and 10 ng/mL of a transforming growth factor-alpha (TGF-α R&D systems 239-a-100) at the same time for 6 hours to induce overexpression of MUC5AC mucus. The PA-LPS and TGF-α were removed, and epidermal growth factors (EGFs) having different concentrations were added to the NCI-H292 cells. Then, the expression of mucus (MUC5AC) gene was identified using a real time polymerase chain reaction (PCR).

The EGFs used herein were recombinant proteins having the same amino acid sequence as natural human epidermal growth factor (hEGF) and synthesized using a gene sequence disclosed in Korean Patent No. 0107023 and WO 94/025592 and an expression vector.

1-2. Anti-Inflammation Effects

MLE-12 cells (ATCC, CRL-2110 murine alveolar epithelial cells) were divided into a 12-well plate such that the number of the MLE-12 cells per well was 5×10⁴. The MLE-12 cells were incubated in a 5% CO₂ incubator at 37° C. for 3 to 4 days. The MLE-12 cells were incubated in a serum free medium for 12 hours, and then treated with EGF and KGF for 1 hour each. The MLE-12 cells were treated with 0.1 U/mL elastase (Sigma, E1250) for 18 hours to induce inflammation. The inflammation was identified by the expression of IntraCellular Adhesion Molecule-1 (ICAM-1) gene using real time PCR.

1-3. Effects on Protecting Cells

MLE-12 cells were divided into a 96-well plate such that the number of MLE-12 cells per well was 5×10⁵. The MLE-12 cells were incubated in 5% CO₂ incubator at 37° C. for one day. The MLE-12 cells were incubated in a medium including 2 U/mL elastase and EGF. A neutral red uptake (NRU) assay was performed using a neutral red that dyes only living cells to measure the degree of cell death. 100 mg/mL of the neutral red dye reagent was added to each well treated with elastase and EGF and the wells were left for 3 hours to dye living cells. The cells were fixed using a fixing solution (1% formaldehyde, 1% CaCl₂) for 1 minute, and the neutral red dye reagent was extracted using a chromogenic solution including 50% ethanol and 1% glacial acetic acid. Then, light absorbance was measured at a wavelength of 540 nm.

1-4. Effects of EGF on Emphysema Animal Model

1-4-1. Animal

6-week old male Sprague-Dawley rats (Japan SLC, Inc) having an average weight ranging from 200 to 250 g were accommodated for 1 week to prepare 7-week old rats. The rats were raised in individual ventilated cage (IVC) racks (GR900 plus rat cage, Polysulfone, 355 W×405 I×230 H mm) at a constant temperature (22±3° C.) at a constant relative humidity (55±15%) using fluorescent lamps (lights are on 08:00 to 20:00) for 12 hours, and food and water were fed ad libitum (solid feed 5L79, Orient Co., Ltd.).

1-4-2. Emphysema Model Using Porcine Pancreatic Elastase (PPE)

120 U/kg, 1 mL/kg of pig pancreas elastase (PPE, Merck 324682) was administered once by intratracheal instillation to rats anesthetized with isoflurane. 1 mL/kg of 0.9% saline was administered once by intratracheal instillation to rats in the group in which emphysema was not induced. In order to uniformly administer test article into lung cavity, the same amount of air as the administered substances was administered during the intratracheal instillation.

After 4 weeks from the administration of PPE, the dose level of EGF 0, 0.1, and 1 mg/kg were respectively administered to the rats by intratracheal instillation. The EGF were administered once a week during 3 weeks and three times in total by intratracheal instillation. After 7 weeks from the PPE administration, the rats were anesthetized with isoflurane, and the lung and the trachea were isolated and fixed using 10% neutral formalin. Then histopathological examination of the lung and the trachea were performed and microscopic images were obtained.

1-4-3. Statistical Analysis

The test results were represented as average ±standard deviation. In order to detect a test group statistically significant from a control group, the results were statistically tested using one way ANOVA, and significance was analyzed using a multiple comparison method.

1-5. Gene Expression Test

The gene expression was identified using the following method.

1-5-1. Isolation of Entire RNA

RNA was isolated using a kit manufactured by QIAGEN (RNeasy Plus Mini Kit, 74134) according to the manuals provided by the manufacturer in order to isolate entire RNA. Absorbance of the isolated RNA was measured using a spectrophotometer at a wavelength of 260 nm to quantitatively analyze RNA.

Complementary DNA (cDNA) was synthesized from the isolated RNA using SuperScript III (Invitrogen, 18080-044). Reverse transcription was performed using 1 μg of RNA sample, 1.25 μM of Oligo(dT) primer, 25 ng of random hexamers, 5 mM of dNTP, 5 mM of MgCl₂, and 200 U SuperScript III. The reverse transcription was performed at 50° C. for 30 minutes to synthesize cDNA and stopped by heating the resultant at 85° C. for 5 minutes. The synthesized cDNA was divided into small amounts and preserved at −70° C.

1-5-2. Preparation of Primer

Primers of human MUC5AC gene (AJ001402) and beta-actin (β-ACT, NM001101) used as an endogenous control of the human MUC5AC gene, and primers of murine ICAM-1 gene (NM010493) and ribosome protein L13 (RPL13, NM016738) used as an endogenous control of the murine ICAM-1 gene were designed based on mRNA base sequence searched from the National Center for Biotechnology Information (NCBI) GeneBank. Primer sets were designed using Primer Express provided by Applied Biosystems, generally according to PCR primer requirements such that the length of the amplified product was about 100 by and Tm was about 80° C. The primers are listed in Table 1 below.

TABLE 1
[Table 1]

| | |
|---|---|
| Human | |
| MUC5AC-for: | 5′CCTCTTCTACCCTGGCGAGAC 3′ |
| | (SEQ ID NO: 2) |
| MUC5AC-rev: | 5′AGCCCATCCTGGTGCTTCT 3′ |
| | (SEQ ID NO: 3) |
| β-ACT-for: | 5′GGCATTGCCGACAGGATG 3′ |
| | (SEQ ID NO: 4) |
| β-ACT-rev: | 5′CTCAGGAGGAGCAATGATCTTGAT 3′ |
| | (SEQ ID NO: 5) |
| Mouse | |
| ICAM-1-for: | 5′GCCACTTCCTCTGGCTGTCA 3′ |
| | (SEQ ID NO: 6) |
| ICAM-1-rev: | 5′CACCGAGTCCTCTTAGCTCTGAG 3′ |
| | (SEQ ID NO: 7) |
| RPL13-for: | 5′AAGGGAGACAGTTCTGCTGAAGAA 3′ |
| | (SEQ ID NO: 8) |
| RPL13-rev: | 5′TTTGTACACATTCCGGATGGG 3′ |
| | (SEQ ID NO: 9) |

1-5-3. Conditions for Reaction

A SYBR green assay was performed by adding cDNA, a forward primer, and a reverse primer to 12.5 mL of a SYBR green PCR master mix (Applied Biosystems, 4309155) such that the total volume of the mixture was set to 25 mL. The amount of cDNA was set to 50 ng based on the amount of total RNA used in RT PCR. The concentration of the primer was determined among the 3×3 concentration combination of 100 nM, 300 nM, and 900 nM of each of the forward primer and the reverse primer by selecting the concentration to efficiently perform amplification without non-specific synthesis. The determined concentrations of the primers for MUC5AC, ICAM-1, RPL1 were respectively 900 nM, and the concentration β-ACT thereof was 300 nM. All test samples were compared with negative control groups without including cDNA to identify whether non-specific amplification was performed. In addition, the same experiments were repeated for 2 to 3 times to reduce experimental errors such as pipetting errors and variations of wells. After the initial PCR performed at 50° C. for 1 minute, at 95° C. for 10 minutes, PCR cycle of 95° C. for 15 seconds and 60° C. for 1 minute was repeated 40 to 50 times. When the PCR was completed, the resultants were dissociated according to a protocol of the manuals to identify whether non-specific amplification occurred.

1-5-4. Measurement of Relative Amount of Gene Expression

The concentration of cDNA was determined such that cycle of threshold (Ct) of a test sample was within the range of a standard curve. The standard curve was obtained using cDNA synthesized after mixing RNA isolated from samples. The standard curve was obtained by repeating experiments for the standard curve three times in the same plate for each reaction and calculating an average Ct obtained from 2 to 3 experiments except for the sample showing significant difference. In this regard, a threshold was adjusted such that a coefficient R2 of the standard curve was close to 1, and an inclination was close to 3.3.

Since the accurate amount of specific genes within cDNA used as the standard is not known, the amount of the gene determined by the current assay is a relative amount based on the standard sample. First of all, the Ct of each sample was obtained using the threshold determined when the standard curve was obtained. The Ct value was compared with the standard curve to obtain the amount of genes. The cDNA concentration variation among the samples was corrected to determine the relative amount of gene expression.

The cDNA concentration may vary according to the efficiency of reverse transcription. That is, even if the reverse transcription is performed under the same conditions, variation in the wells in the PCR device may cause variation in the amount of synthesized cDNA. Since isolation of only cDNA or measurement of the concentration of cDNA was not performed after the reverse transcription, the cDNA concentration variation by the RT efficiency was corrected using an endogenous control. The endogenous control was house keeping gene RPL13. Since it was assumed that the amounts of expressed endogenous control were the same, the relative amount of cDNA is proportional to the amount of expressed endogenous control. Thus, a normalization fold to correct the cDNA variation is a reciprocal number of the relative expression amount of endogenous control.

When the cDNA concentration variation was corrected, the relative amount of gene expression was obtained by multiplying the amount of gene obtained by the standard curve by the normalization fold.

### 2. RESULTS

2-1. Effects of EGF on In Vitro Chronic Bronchitis Model

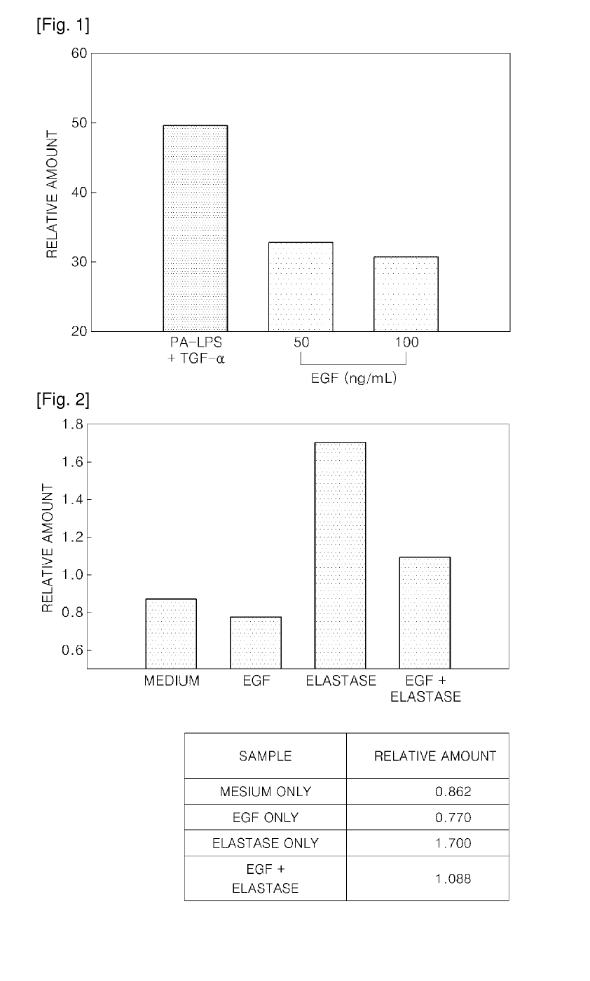

A mucus gene, MUC5AC was overexpressed by treating NCI-H292 cells simultaneously with 100 mg/mL of PA-LPS and 10 ng/mL of TGF-α and the NCI-H292 cells were treated with 50 and 100 ng/mL of EGF. Then, the expression of mRNA of MUC5AC was observed, and the results are shown in FIG. 1.

As shown in FIG. 1, the expression of MUC5AC was reduced by 65% using 50 ng/mL of EGF, and the expression of MUC5AC was reduced by 60% using 100 ng/mL of EGF. Thus, the effects of EGF on reducing the expression of mucus were identified.

2-2. Effects of EGF on In Vitro Emphysema Model

2-2-1. Anti-inflammation Effects of EGF

ICAM-1 gene expression in TMLE-12 cells treated with 0.1 U/mL of elastase was compared with that in TMLE-12 cells treated with 0.1 U/mL of elastase and 100 ng/mL of EGF, and the results are shown in FIG. 2. ICAM-1 is known to bring macrophages and neutrophils into lung wounds thereby worsening inflammation. The TMLE-12 cells were pre-treated with EGF for 1 hour and treated with elastase for 18 hours to observe anti-inflammation effects of EGF.

As shown in FIG. 2, the expression of ICAM-1 gene in the group treated with the elastase increased by twice compared with that of the negative control group. The expression of ICAM-1 gene was reduced by over 60% by pre-treating the cells with the EGF. Thus, anti-inflammation effects of the EGF were identified.

2-2-2. Effects on Protecting Alveolar Cells

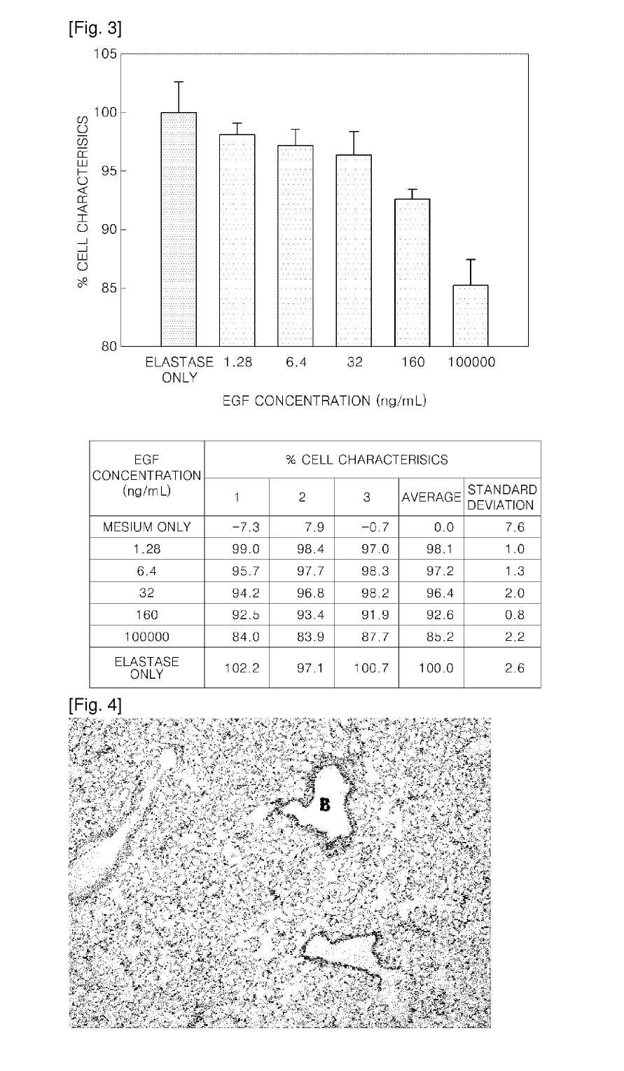

Cell toxicity of MLE-12 cells treated with 2 U/mL of elastase was compared with cell toxicity of MLE-12 cells treated with various concentrations of EGF, and the results are shown in FIG. 3.

As shown in FIG. 3, a group treated with elastase only was regarded as having 100% cell toxicity, and a group treated with a culture medium only was regarded as having 0% cell toxicity. Effects of the EGF on protecting the cells were observed. As a result, cell toxicity was reduced by about 10% by adding 160 ng/mL of the EGF and by about 15% by adding 100 μg/mL of the EGF. Thus, it was identified that EGF has an effect on protecting cells from cell death by elastase.

2-2-3. PPE-Induced Emphysema Model

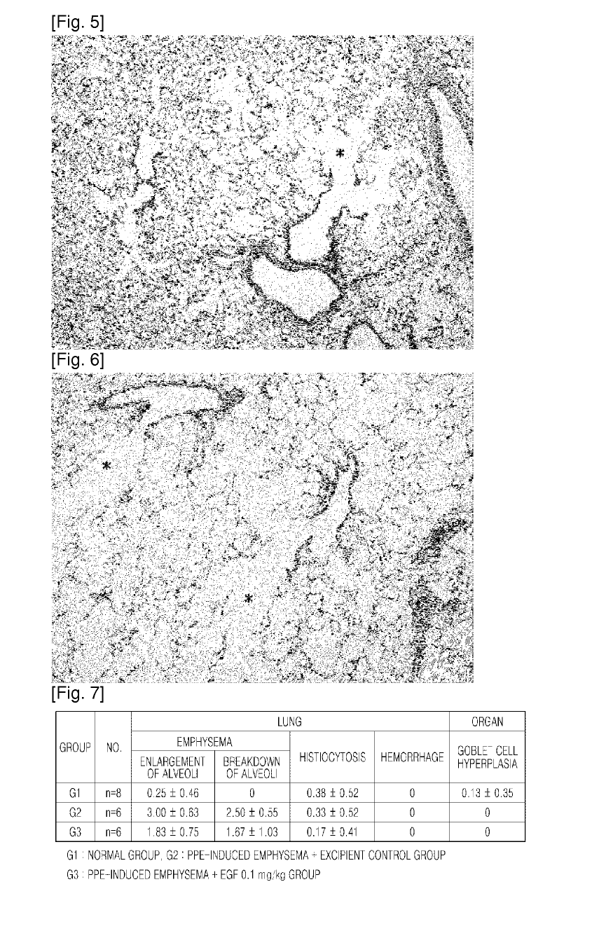

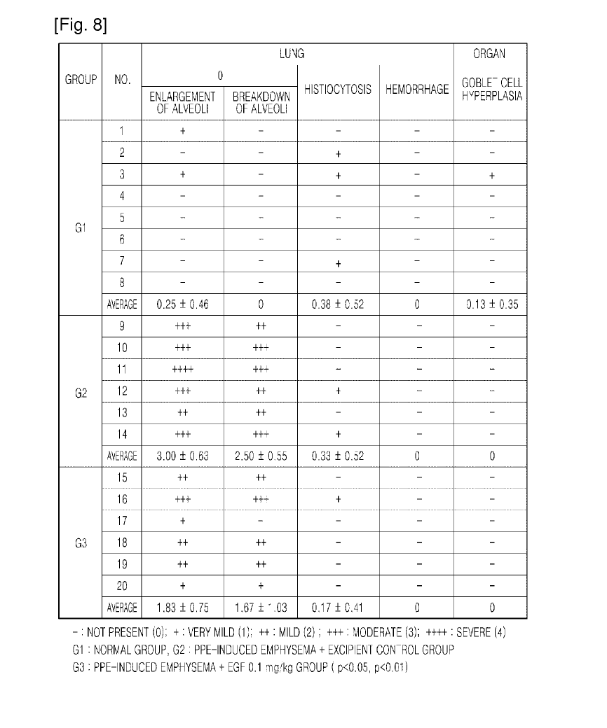

In order to identify the efficacy of EGF on emphysema model of rats induced by PPE, histopathological examination was performed in tracheas and lungs. FIGS. 4, 5, and 6 illustrate optical microscopic images (×40) of tracheas isolated from a normal group (G1), a control group of PPE-induced emphysema model (G2), and a group of PPE-induced emphysema model treated with 0.1 mg/kg of EGF (G3) (FIG. 4: The number of individuals is 5, FIG. 5: The number of individuals is 20, and FIG. 6: The number of individuals is 11). In addition, FIGS. 7 and 8 illustrated the results of the tissue pathological tests of tracheas and lungs with a table.

As a result of histopathological examination, 2 cases showed enlargement of alveoli, 3 cases showed histiocytosis, and 1 case showed goblet cell hyperplasia in the normal group (G1).

In the groups of PPE-induced emphysema model (G2 and G3), enlargement of alveoli and breakdown of alveoli, as emphysematous lesions, were mainly observed. In some cases, alveolar histiocytosis having histocyte plenty of large cytoplasm with vacuolation in the alveolar cavity and local hemorrhage were observed.

The control group of PPE-induced emphysema model (G2) showed various degrees of enlargement of alveoli and breakdown of alveoli from mild to severe. The lesions of G2 are more serious than the other groups. 1 case showed severe enlargement of alveoli, 4 cases showed moderate enlargement of alveoli, and 1 case showed mild enlargement of alveoli, 3 cases showed moderate breakdown of alveoli and 3 cases showed mild breakdown of alveoli, and 2 cases showed histiocytosis, but the degree was mild.

In the group of PPE-induced emphysema model treated with 0.1 mg/kg of EGF (G3), 1 case showed moderate enlargement of alveoli, 3 cases showed mild enlargement of alveoli, and 2 cases showed very mild enlargement of alveoli, 1 case showed moderate breakdown of alveoli, 3 cases showed mild breakdown of alveoli, and 1 case showed very mild breakdown of alveoli, and 1 case showed very mild histiocytosis.

### 3. CONCLUSION AND CONSIDERATION

As a result of observing the effects of EGF on chronic bronchitis and emphysema using in vitro evaluation method, the expression of the gene of MUC5AC that is representative mucin overexpressed by PA-LPS and TGF-α was relatively reduced by the administration of EGF, and after the EGF treatment, the expression of ICAM-1 was reduced in alveolar cells in which inflammation was induced. Thus, it was identified that the EGF suppresses mucus hypersecretion in airways.

The destruction of alveolar cells that is symptom of emphysema was induced using elastase, and living cells were dyed with neutral red. The cell death induced by the elastase was reduced by administering the EGF. Thus, the effects of the EGF on protecting MLE-12 cells, as alveolar cells, were identified.

In order to identify the efficacy of EGF on PPE-induced emphysema model, histopathological examination was performed in tracheas and lungs, As a result of histopathological examination. Emphysematous lesions such as enlargement of alveolar cavity and breakdown of alveolar walls were mainly observed, and hemorrhaging and inflammation of lungs, or abnormal changes such as in airway epithelium were not significantly observed in PPE-induced emphysema model.

These are similar to conventional reports indicating the lung lesions induced by human neutrophil elastase includes an acute phase showing lung hemorrhaging and neutrophil infiltration and a chronic phase showing hyperplasia of lung, degradation of elastic recoil, and airspace enlargement.

According to the results, the normal group (G1) showed 4 cases of very mild enlargement of alveoli in the aspects of the lesion distribution and its severity, which is different from lesions of the groups of PPE-induced emphysema model. The very mild enlargement of alveoli is considered to be caused by physical stimulation of the substance while being injected into the tracheas rather than the influence of the substance itself. The severe emphysematous lesions were observed in the control group of PPE-induced emphysema model. It was identified that the emphysematous lesions were induced by the elastase. The degree and the number of enlargement and breakdown of alveoli were reduced in the group with emphysema treated with EGF (G3) compared with the control group of PPE-induced emphysema model. Thus, it was considered that the emphysematous lesions were relieved by administering the EGF in the PPE-induced emphysema model as a result of histopathological examination.

In summary, the EGF has effects on suppressing mucus hypersecretion, protecting alveolar cells, and improving the tissue pathological manifestation in emphysema model. Thus, the EGF has effects on preventing or treating not only COPDs such as chronic bronchitis, emphysema, but also mucus hypersecretion in airways in a variety of chronic pulmonary diseases.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A pharmaceutical composition for preventing or treating mucus hypersecretion in airways, the pharmaceutical composition comprising an epidermal growth factor (EGF) and a pharmaceutically acceptable carrier.

2. A pharmaceutical composition for preventing or treating a chronic obstructive pulmonary disease (COPD), the pharmaceutical composition comprising an epidermal growth factor (EGF) and a pharmaceutically acceptable carrier.

3. The pharmaceutical composition of claim 2, wherein the chronic obstructive pulmonary disease is chronic bronchitis or emphysema.

4. The pharmaceutical composition of claim 1, wherein the EGF is chemically or recombinantly synthesized or derived from a natural source.

5. The pharmaceutical composition of claim 1, wherein the pharmaceutically acceptable carrier comprises saline or a buffer solution.

6. A package for preventing or treating mucus hypersecretion in airways, the package comprising an inhaler device and a fluidable preparation comprising an epidermal growth factor (EGF) that is contained in the inhaler device and is pharmaceutically active.

7. The package of claim 6, wherein the EGF is chemically or recombinantly synthesized or derived from a natural source.

8. The package of claim 6, wherein the inhaler device is a meter-dose inhaler, and the EGF is mixed with a propellant.

9. The package of claim 6, wherein the inhaler device comprises a spray, and the EGF is mixed with saline or a buffer solution.

10. The pharmaceutical composition of claim 2, wherein the EGF is chemically or recombinantly synthesized or derived from a natural source.

11. The pharmaceutical composition of claim 2, wherein the pharmaceutically acceptable carrier comprises saline or a buffer solution.

12. The package of claim 7, wherein the inhaler device is a meter-dose inhaler, and the EGF is mixed with a propellant.

13. The package of claim 7, wherein the inhaler device comprises a spray, and the EGF is mixed with saline or a buffer solution.
